(19) United States
(12) Patent Application Publication
(10) Pub. No.: US 2003/0017558 A1
(43) Pub. Date: Jan. 23, 2003

(54) METHOD FOR MIXOTROPHIC CULTURE OF SPIRULINAS FOR PRODUCING A BIOMASS RICH IN OMEGA W6 POLYUNSATURATED FATTY ACIDS AND/OR IN SULPHOLIPIDS

(76) Inventors: QUOC KIET PHAM, CERGY SAINT CHRISTOPHE (FR); HUBERT DURAND-CHASTEL, 75013 PARIS (FR)

Correspondence Address: DENNISON MESEROLE POLLOCK & SCHEINER, 1745 JEFFERSON DAVIS HIGHWAY, SUITE 612, ARLINGTON, VA, 22202

(21) Appl. No.: 09/284,961

(22) PCT Filed: Sep. 16, 1998
(86) PCT No.: PCT/FR98/01977

(30) Foreign Application Priority Data
Sep. 19, 1997 (FR) 97/11678

(51) Int. Cl. C12P 7/64

(52) U.S. Cl. 435/134

(57) ABSTRACT

The invention relates to a method for mixotrophic culture of Spirulina for producing a biomass rich in ω6 polyunsaturated fatty acids, in particular rich in γ-linolenic acid, and/or in sulfolipids, comprising at least one culture stage of Spirulina in the presence of ammonium linoleate and optionally of glucose.

The invention relates to a method for mixotrophic culture of Spirulina for producing a biomass rich in biologically active compounds, particularly rich in ω6 polyunsaturated fatty acids (especially γ-linolenic acid) and/or in sulfolipids.

Spirulina have been dicovered and used as food supplement for human body for several centuries. They are precious and rare blue-green microalgae which have a particular nutritional value for children suffering from malnutrition [1-5]. They develop mainly in alkaline water of certain tropical lakes in arid zone. They are rich in compounds that have nutritional and biomedical interest: essential amino acids, vitamins (A, B12, E . . . ), essential polyunsaturated fatty acids (especially γ-linolenic acid) recognized as precursors of eïcosanoids (prostaglandins PGE1, PGE2 and thromboxane TXB2 . . . ) in mammals [6]. The eicosanoids (PGE2) and leukotrienes are implied in immune reactivity and inflammatory reaction. The prostaglandin PGE2 inhibits the proliferation of T4 lymphocytes which take part in the surveillance of cancerous cells.

The present invention concerns a culture process of Spirulina in order to obtain a biomass rich in γ-linolenic acid and/or in antiviral sulfolipids [7].

Spirulina are blue-green algae belonging to the phylum: Cyanophyta, class Cyanophyceae, order: Nostocales, family: Oscillatoriaceae, genus: Spirulina or Arthrospira.

Different strains of Spirulina exist, notably the species platensis and Spirulina maxima (Bourrelly P. 1970. Les algues bleues ou cyanophycées, in “Les Algues d'eau douce, Tome III, Editions N. Boubée).

It was now been found that particular conditions of culture, using ammonium linoleate as a supplement under optimized temperature and illumination conditions, allowed the obtention of a Spirulina biomass particularly rich in bioactive compounds.

Spirulina grow fairly well in culture media supplemented with ammonium linoleate. They absorb exogenous linoleic acid under the form of ammonium linoleate in order to synthesize γ-linolenic acid in their lipids such as monogalactosyldiacylglycerol (MGDG), digalactosyldiacylglycerol (DGDG), sulfoquinovosyldiacylglycerol (SQDG) and phosphatidylglycerol (PG).

The present invention therefore concerns a method for mixotrophic culture of Spirulina for producing a biomass rich in ω6 polyunsaturated fatty acids, especially γ-linolenic acid, and/or in sulfolipids, characterized in that this production comprises at least one culture stage of Spirulina in the presence of ammonium linoleate.

The concentration of ammonium linoleate added to the medium is comprised between 30 and 90 μM/l with a preferable range of about 35-75 μM/l.

Advantageously, the culture stage of Spirulina in the presence of ammonium linoleate is carried out at a temperature range of 20 to 35° C. and under an illumination between 50 and 125 μE/m²/s, preferably between 75 and μE/m²/s according to an illumination cycle by 24 h, with a preferable range of about 8-12 h of white light and 16-12 h in obscurity.

In a particularly preferred aspect, the illumination cycle is of about 12 h of white light and 12 h in obscurity.

The ammonium linoleate is advantageously added to the culture medium is realised during the exponential phase of growth, preferably in the middle of this phase, advantageously when the optical density (O.D.) of the medium which indicates the cellular concentration is approximately equal to 2.

Preferably, when the production of Spirulina biomass rich in ω6 polyunsaturated fatty acids, especially rich in ε-linolenic acid, is carried out, the culture temperature is between about 24° C. and 35° C., advantageously about 30° C. and the illumination is preferably comprised between 75 and 125 μE/m²/s, advantageously between 75 and 100 μE/m²/s, preferably according to an illumination cycle by 24 h of about 8-12 h of white light and 16-12 h in obscurity.

In a particularly preferred aspect, the illumination cycle is of about 12 h of white light and 12 h in obscurity.

When it is desired to produce a Spirulina biomass rich in sulfolipids, the culture temperature preferably is between about 20° C. and 25° C., and the illumination is preferably between about 100 and 125 μE/m²/s, advantageously according to an illumination cycle by 24 h of about 8-12 h of white light and 16-12 h in obscurity.

In a particularly preferred aspect, the illumination cycle is about 12 h of white light and 12 h in obscurity.

In an advantageous aspect of the invention, the culture medium may be simultaneously supplemented with glucose, preferably during the exponential phase of growth, at a concentration of glucose of about 2 to 5 g/l, preferably 2,5 to 3 g/l.

This supplementation can be advantageously carried out at a temperature of about 30° C., under an illumination of about 50 to 100 μE/m²/s, according to an alternate cycle by 24 h of about 8-12 h of white light and 16-12 h in obscurity, preferably 12 h of white light and 12 h in obscurity.

The Spirulina biomass can be produced in a racepond or in a sterile photobioreactor. The ponds and different photobioreactors which are appropriate for this type of culture are well known to skilled persons and described by several authors [12,13].

The production of a Spirulina biomass rich in ω6 polyunsaturated fatty acids, particularly rich in γ-linolenic acid and/or in sulfolipids in pond or photobioreactor can be carried out in 3 stages, namely:

1) preservation of original strains of Spirulina and multiplication of these strains (preparation of preculture or inoculum for production),

2) mass production (mass culture or production in large scale), and

3) harvest of Spirulina biomass produced.

I. Strains of Spirulina Used for Production:

According to the invention, the culture process applies to all existing wild strains of Spirulina (i.e, non-mutant strains), notably the strains described in the publications cited above. For example, the strain used for production can be chosen among the following strains:

Spirulina platensis PCC 8005 from Institut Pasteur, Paris;

Spirulina maxima and Spirulina texcoco (Texcoco, Mexico);

Spirulina crater (Laboratoire La Roquette, France).

The general chemical composition of these strains was determined by well known methods of analysis [8-12] and presented in the following tables 1, 2 and 3:

TABLE 1
General chemical composition (%)

| | Sp. 8005 | Sp. Maxima | Sp. Texcoco | Sp. crater |
|---|---|---|---|---|
| Proteins | 56-61 | 59-60 | 60-61 | 58-60 |
| Carbohydrates | 16-19 | 18-19 | 13-14 | 12-14 |
| Lipids | 6.7-6.9 | 6.3-6.4 | 6.0-6.3 | 5-6 |
| Minerals | 7-13 | 7.5-8 | 6.0-12 | 7-8 |
| Humidity | 7-8 | 7.7-8 | 6.8-7 | 7-8 |
| Carotenoids | 0.12-0.22 | 0.25-0.27 | 0.10-0.14 | 0.12-0.15 |
| Chlorophyll a | 0.7-0.8 | 0.77-0.80 | 0.70-0.76 | 0.70-0.78 |

TABLE 2
Lipid composition (%)

| | Sp. 8005 | Sp. Maxima | Sp. Texcoco | Sp. crater |
|---|---|---|---|---|
| MGDG | 31.5-33 | 34.0-38.9 | 34-38 | 35-39 |
| DGDG | 15.7-17.3 | 15.0-17.8 | 14.0-17.5 | 16-18 |
| PG | 24.8-26.2 | 22.0-23.4 | 25.0-26.7 | 22.0-23.6 |
| SQDG | 24.8-26.6 | 22.3-23.7 | 24-26 | 22.0-24.5 |

TABLE 3
Total fatty acid composition (%)

| | Sp. 8005 | Sp. Maxima | Sp. Texcoco | Sp. crater |
|---|---|---|---|---|
| Palmitic acid | 51.2 | 51.4 | 50.6 | 49.2 |
| (C16:0) | | | | |
| Palmitoleic acid | 3.6 | 5.0 | 4.3 | 2.1 |
| (C16:1) | | | | |
| Stearic acid | 0.7 | 1.0 | 1.5 | 2.7 |
| (C18:0) | | | | |
| Oleic acid | 5.9 | 7.3 | 7.2 | 7.0 |
| (C18:1) | | | | |
| Linoleic acid | 18.2 | 14.6 | 15.0 | 18.5 |
| (C18:2) | | | | |
| γ-linolenic acid | 20.4 | 20.7 | 21.4 | 20.5 |
| (γ-C18:3) | | | | |
| ΣC16/ΣC18 | 54.8/45.2 | 56.4/43.6 | 54.9/45.1 | 51.3/48.7 |

In table 3 above, the saturated and unsaturated fatty acids are designated as follows:

C16:0: palmitic acid (or hexadecanoic acid)

C16:1 (Δ⁹ cis C16:1): palmitoleic acid (or cis-9-hexadecenoic acid)

C18:0: stearic acid (or octadecanoic acid)

C18:1 n-9 (Δ⁹cis C18:1): oleic acid

C18:2 n-6 (Δ^{9,12}cis,cis C18:2): linoleic acid (or cis, cis-9,12-octadecadienoic acid)

C18:3 n-6 (Δ⁶,^{9,12}cis,cis,cis C18:3): γ-linolenic acid (or cis,cis,cis-6,9,12-octadecatrienoic acid).

II. Preservation of Original Strains:

The preservation of original strains was realised from strains called “preservation strains” which were selected and preserved in the following way:

a) Zarrouk's sterile culture medium having a chemical composition similar to that cited in table 4 below;

b) Temperature of culture of about 20 to 22° C.;

c) Alkaline culture medium corresponding to a pH equal to about 8.5;

d) Relatively weak illumination for the preservation (about from 50 to 75 μE/m²/s);

e) Alternate illumination cycle of about 16 h of white light and 8 h in obscurity by 24 h.

f) In the case of the inoculum preparation for mass production, the broth of preliminary precultures was maintained under aeration and general turbulence by bubbling air enriched with 1% in volume of CO₂ at a flow rate of about 20-30 liters/liter of culture/hour. Thanks to the slow growth of preservation strains in Zarrouk liquid medium at 22° C., under an illumination of 50 to 75 μE/m²/s, these strains can be regularly planted out after 15-20 days in order to reserve permanently an adequate quantity of inoculum.

g) In the case of preservation of original strains, these strains were kept at 22° C. under illumination from 50 to 75 μE/m²/s during 2-3 months without aeration.

h) the pH of Zarrouk's medium was adjusted to 8.5 before sterilisation of the medium performed at 120° C. for 20 minutes.

TABLE 4
Chemical composition of Zarrouk's medium (1966)

| | | |
|---|---|---|
| NaHCO₃ | 16.8 | g |
| K₂HPO₄ | 0.5 | g |
| NaNO₃ | 2.5 | g |
| MgSO₄.7H₂O | 0.2 | g |
| KSO₄ | 1.0 | g |
| NaCl | 1.0 | g |
| CaCl₂ | 40 | mg |
| FeSO₄ | 10 | mg |
| EDTA | 80 | mg |
| Solution A5* | 1 | ml |
| Solution B6** | 1 | ml |
| H₂O | 1 | liter |

*1 liter of solution A5 contains:

| | | |
|---|---|---|
| H₃BO₃ | 2.86 | g |
| MnCl₂.4H₂O | 1.81 | g |
| ZnSO₄.7H₂O | 0.2 | g |
| CuSO₄.5H₂O | 79 | mg |
| MoO₃ | 15 | mg |
| (or NaMoO₄) | 21 | mg |

**1 liter of solution B6 contains:

| | | |
|---|---|---|
| NH₄VO₃ | 23 | mg |
| K₂Cr₂(SO₄)₄.24H₂O | 96 | mg |
| NiSO₄.7H₂O | 48 | mg |
| Na₂WO₄.2H₂O | 18 | mg |
| Ti₂(SO₄)₃ | 40 | mg |
| Co(NO₃)₂.6H₂O | 44 | mg |

III. Mass Production:

In the following description, the number of stages or phases of preculture or culture as well as their duration is only mentioned as an indication as they, of course, can vary according to the used conditions, notably the initial cellular concentration.

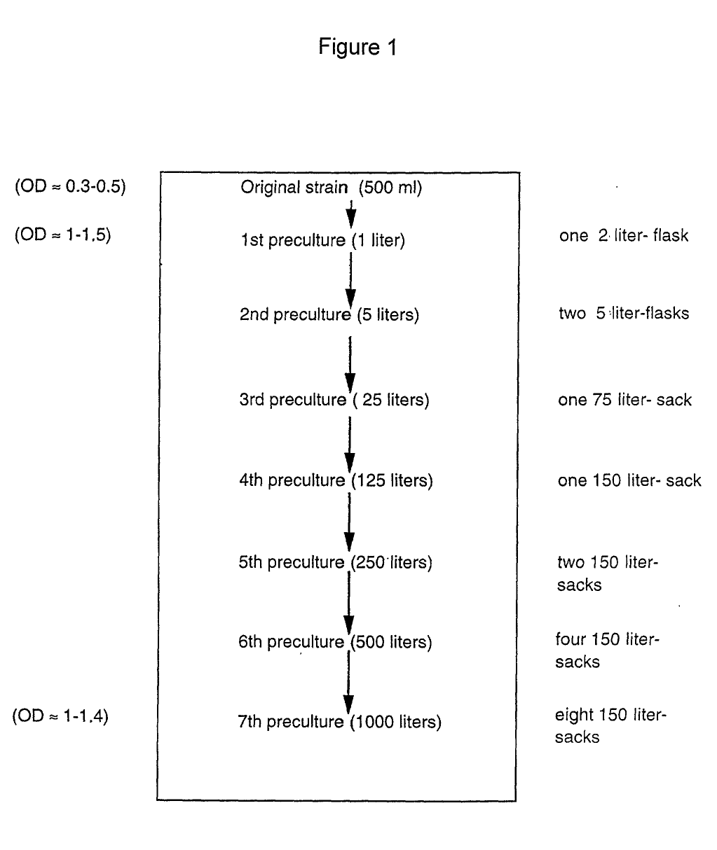

For the mixotrophic production of a Spirulina biomass rich in ω6 polyunsaturated fatty acids, particularly rich in γ-linolenic acid and/or in sulfolipids, the preparation phase of precultures, also called the preparation phase of inoculum (multiplication of strains), can be carried out in 7 successive stages as indicated on FIG. 1.

It is noticeable that this preparation phase of the inoculum is performed only once at the beginning of the production method. For the preparation of the final inoculum, either the inoculum obtained at the last stages of preculture (6th or 7th stage) or a part of the Spirulina strains harvested at the end of mass production can be used for innoculation of the medium at an earlier stage, which represents a saving of time and of feasibility at the industrial scale.

An important parameter, independently of the number of phases, is the measure of the cellular concentration by the optical density (O.D.) at 560 nm, which allows an estimation of the cellular dry weight.

Measuring the optical density allows determining the initial cellular concentration and optimizing the culture conditions: by using a high initial cellular concentration, the culture duration can be decreased while simultaneously increasing the culture yield. This is particularly advantageous for the production at the industrial scale.

This measure simultaneously allows determining the growth stages of the strain and then to determine the moment when ammonium linoleate is added to the culture medium (at this moment, O.D. is about 2).

The biomass is estimated at this wavelength located at a hollow of the absorption spectrum and which does not correspond to any particular pigment. The absorbance at this wavelength, as far as it does not exceed a value of 2, varies linearly with the cellular concentration. The standardization curve shows that the optical density at 560 nm, which varies with cellular volumes and cellular concentration, is linearly proportional to cellular dry weights: O.D.=1≈0.7 g dry/liter.

When the initial multiplication of strains (preparation of precultures) is carried out in 7 stages, it is possible from the 5th stage on to use new ZK sterile media of which the compositions are given hereafter in table 5, instead of Zarrouk's medium.

The ZK media from (1) to (4) are new media particularly adapted to carry out the culture process of Spirulina described above, notably at the industrial scale and represent a later object of invention. The ZK (1) medium is particularly preferred.

TABLE 5
Chemical composition of ZK media from (1) to (4)

| ZK (1) | | | ZK (2) | | |
|---|---|---|---|---|---|
| NaHCO₃ | 8.4 | g | NaHCO₃ | 8.4 | g |
| NaCl | 5.0 | g | NaCl | 4.5 | g |
| K₂HPO₄ | 0.2 | g | K₂HPO₄ | 0.2 | g |
| MgSO₄.7H₂O | 0.2 | g | NaNO₃ | 3.0 | g |
| KNO₃ | 2.5 | g | MgSO₄.7H₂O | 0.2 | g |
| FeSO₄ | 0.01 | g | FeSO₄ | 0.01 | g |
| or FeSO₄.7H₂O | 0.018 | g | H₂O | 1 | liter |
| H₂O | 1 | liter | | | |
| ZK (3) | | | ZK (4) | | |
| NaHCO₃ | 8.4 | g | NaHCO₃ | 8.4 | g |
| NaCl | 5.5 | g | NaCl | 5.5 | g |
| K₂HPO₄ | 0.3 | g | (NH₂)₂CO or (NH₄)₂SO₄ | 0.1 | g |
| MgSO₄.7H₂O | 0.2 | g | NH₄H₂PO₄ | 0.1 | g |
| (NH₂)₂CO or | 0.2 | g | K₂SO₄ | 0.2 | g |
| (NH₄)₂SO₄ | | | MgSO₄.7H₂O | 0.2 | g |
| FeSO₄ | 0.01 | g | FeSO₄ | 0.01 | g |
| H₂O | 1 | liter | H₂O | 1 | liter |

Culture conditions which are particularly advantageous to the production of Spirulina biomass rich in γ-linolenic acid and/or in sulfolipids consist in:

bubbling the precultures with 60 to 80 liters of sterile air enriched with 1% of CO₂/liter of culture/hour;

maintaining the pH of the culture medium between 8.5 and 10.5 during the growth, preferably 9 to 10, in order to avoid contamination by other microorganisms which are not able to develop in a very basic medium;

maintaining the minimum concentrations of nitrate, phosphate and hydrogenocarbonate adapted to the needs of the Spirulina strain during the growth;

maintaining the culture temperature at 30° C. while providing an illumination of about 100 to 125 μE/m² is during about 8-12 h of white light and about 12-16 h in obscurity by 24 h during the growth, then lowering the light intensity to 75-100 μE/m²/s during the last phase of production.

In an advantageous aspect, the concentrations of ions such as hydrogenocarbonate, phosphate and nitrate covering the needs of Spirulina strains during the growth phase are the following ones:

concentration of sodium hydrogenocarbonate of about 8 to 10 g/l in early exponential phase of growth and about 0.8 to 0.9 g/l in stationary phase of growth;

concentration of dipotassium hydrogenophosphate of about 0.2 g/l in early exponential phase of growth and about 0.02 g/l in stationary phase of growth;

concentration of sodium nitrate or potassium nitrate of about 2.5 g/l and concentration of nitrate NO₃⁻ about 0.45 to 1 g/l.

III.1-Production of Biomass in Pond:

a) Production of a Biomass Rich in γ-Linolenic Acid:

The production of a Spirulina biomass rich in γ-linolenic acid in pond can be carried out for example in 5 successive phases, in each of which a pricking from a well known culture quantity of the previous phase in a determined quantity of new medium (Zarrouk's medium or ZK medium) is carried out, from an inoculum prepared at a previous phase (phase 0).

The growth is measured by the optical density (O.D.) at 560 nm.

Advantageously, a supplementation with ammonium linoleate is carried out during the exponential phase of growth, for example in the middle of this growth phase, under an illumination of 75 to 100 μE/m² is. This phase is preferably carried out in a new 10 m³-pond while the 4 first phases have taken place in a first 45 m³-pond. The pond is preferably equipped with a mixing circulating system, for example a paddlewheel, in order to ensure the circulation of culture medium. An alternate illumination of about 8-12 h of white light and about 16-18 h in obscurity by 24 h during 72-96 h is used for stimulating the synthesis of γ-linolenic acid in Spirulina cells. Moreover, after the supplementation with linoleate, the bubbling of sterile air enriched with 1% of CO₂ is lowered, if necessary for maintenance of pH, and the bubbling is maintained at a flow rate of 30-40 l/liter of culture/h during 24 h in order to avoid the formation of foam, then at a higher flow rate of 50 to 70 l/l of culture/h.

b) Production of Biomass Rich in Sulfolipids:

The production of Spirulina biomass rich in sulfolipids in pond can be, for example, carried out in a 45 m³-pond in 6 successive phases of which the 4 first phases are carried out in the same way as for obtaining a Spirulina biomass rich in γ-linolenic acid. During the exponential phase of growth, the culture is preferably carried out in a second 12 m³-pond and maintained at about 30° C. under an illumination of 75 to 100 μE/m²/s during 48 h, then at 24° C. under a higher illumination of 100 to 125 μE/m²/s during 48-72 h. Simultaneously, the illumination cycle by 24 h should be preferably regulated to about 8-12 h of white light/16-12 h in obscurity, preferably of 12 h of white light/12 h in obscurity, in order to stimulate the synthesis of sulfolipids in Spirulina cells.

The culture is then preferably maintained at a temperature of 20 to 22° C., under an illumination of 100 to 125 μE/m²/s, with an alternate cycle of illumination of about 8-12 h of white light/16-12 h in obscurity during 72-96 h before harvesting the biomass.

Moreover, the bubbling of air enriched with 1% of CO₂ is advantageously lowered and maintained at a flow rate of 25 l/l of culture/h during 24-48 h, then at a higher flow rate from 40 to 60 l/l of culture/h during 48-96 h after the supplementation with linoleate, if necessary to maintain the pH.

The supplementation with ammonium linoleate is carried out during this exponential phase of growth which can be for example the fifth stage of production.

The harvest of the Spirulina biomass rich in γ-linolenic acid is carried out at the end of the exponential phase of growth while the harvest of the Spirulina biomass rich in sulfolipids is done during the stationary phase. The optical density at this moment is about from 3 to 4. The biomass is maintained in several decanting tanks at 24° C. during 24 h in order to eliminate the supernatant.

The harvest can be carried out for example as follows:

The biomass which has precipitated at the bottom of decanting tanks is filtered or submitted to a centrifugation at 5000 rounds/min during 15 minutes, then rinsed with a solution of NaCl at concentration of 10 g NaCl/liter a 24° C. in order to eliminate an important part of residual salts and of traces of linoleate present in the culture medium. Moreover, the salt concentration of the rinsing solution, which is similar to the one of the culture medium, permits to avoid a breaking of the cellular membranes due to osmotic pressure. Then, a new centrifugation is carried out at 5000 rounds/min during 15 minutes and the biomass is finally rinsed with distilled or demineralized water (3 times) at 20-24° C. The harvest of the Spirulina biomass is preferably carried out at a temperature comprised between 20 and 24° C. in order to protect the transformation of linoleic acid into γ-linolenic acid. This low temperature also makes it possible to avoid the cellular degradation and the lipid denaturation.

The obtained biomass can be lyophilized or sprayed, preferably immediately, in order to avoid a secondary contamination.

The supernatant can be recycled for a new culture (6th phase) provided that the concentrations of bicarbonate, nitrate, phosphate, potassium, sodium, linoleate are re-controlled and that the medium is re-completed with nutritive elements which are necessary to the growth of Spirulina.

These needs are about 470 g of carbon; 120 g of nitrogen; 13.3 g of potassium; 7.6 g of phosphate; 5.25 g of sulfur; 4 g of chlorine; 1.4 g of magnesium; 1 g of calcium; 0.47 g of iron and 0.3 g of sodium in order to synthesize 1 kg of biomass. According to these needs, the amounts of the necessary minerals can be calculated in order to re-complete the medium after the harvest of a known quantity of biomass.

III.2-Production of Biomass in a Photobioreactor:

The biomass of Spirulina rich in γ-linolenic acid and/or in sulfolipids can be alternatively produced in a sterile photobioreactor.

This type of culture is particularly advantageous because it allows the obtention of a Spirulina biomass containing a still more important quantity of γ-linolenic acid or sulfolipids as compared with a culture in pond. Moreover, the control of culture conditions and the culture in a closed system make it possible to ensure a high purity of the finished product, particularly fit to pharmaceutical or cosmetic uses.

The preparation of inoculum (multiplication of Spirulina strains) can be for example carried out in 4 successive stages, in each of which a known quantity of the culture of the previous stage is planted out in a determined quantity of new medium, preferably Zarrouk's medium.

For example, the first 3 precultures can be carried out in 50 ml-, 250 ml- and 1 liter-flasks; and the 4th preculture in a 2 liter-photobioreactor. The preferred conditions of culture are detailed hereafter in the examples.

a) Production of a Biomass Rich in γ-Linolenic Acid:

For obtaining a Spirulina biomass rich in γ-linolenic acid, the production can be for example carried out in 2 phases, in a photobioreactor, preferably in 2 different 7 liter-photobioreactors.

The cellular growth is also measured by the O.D. at 560 nm.

The incorporation of ammonium linoleate is preferably performed during the exponential phase of growth, at a concentration of about 30 to 90 μM/l, notably of 35 to 75 μM/l, preferably during 4-6 days.

The culture is then preferably carried out according to the following conditions:

temperature of about 28 to 32° C., preferably 30° C.;

illumination of about 75 to 100 μE/m²/s, according to a cycle by 24 h of 8 to 12 h of white light/16 to 12 h in obscurity after supplementation with ammonium linoleate;

bubbling of air enriched with 1% of CO₂ at a flow rate about of 20 to 30 l/l of culture/h during 24 h after supplementation with ammonium linoleate and then at a higher flow rate of 50 to 70 l/l of culture/h.

The stirring speed during the production is preferably of 100 to 150 rounds/min.

The harvest is carried out after 4 to 6 days of culture under the conditions indicated above for the cuture of Spirulina in a pond.

b) Production of a Biomass Rich in Sulfolipids:

For obtaining a biomass of Spirulina rich in sulfolipids in a photobioreactor, the production can be for example carried out in 2 phases:

During the second phase, the medium is supplemented with ammonium linoleate in the same conditions as for obtaining a biomass of Spirulina rich in γ-linolenic acid.

This phase then preferably takes place during 5 to 8 days in 3 successive sub-phases under the following conditions:

1st sub-phase

Temperature of 28 to 32° C., preferably 30° C.;

Illumination of 75 to 100 μE/m²/s, according to a cycle of 8 to 12 h of white light 16 to 12 h in obscurity by 24 h during 48 h;

Bubbling of air enriched with 1% of CO₂ at a flow rate of 25 to 35 l/l of culture/h during 24 to 48 h;

2nd sub-phase:

Temperature of 20 to 25° C., preferably 24° C.;

Illumination of 100 to 125 μE/m²/s according to the same cycle as in the first sub-phase;

Bubbling of air enriched with 1% of CO₂ at a flow rate of 40 to 50 l/l of culture/h during 48 to 72 h;

3rd sub-phase:

Temperature of 20 to 22° C.;

Illumination of 100 to 125 μE/m²/s with a light cycle according to the same cycle of light as in first sub-phase during 72-96 h;

Bubbling of air enriched with 1% of CO₂ at a flow rate of 40 to 60 l/l of culture/h during 72-96 h.

The harvest of the Spirulina biomass rich in sulfolipids is carried out under the above-described conditions.

The culture processes described above, in pond or in sterile photobioreactor, allow the obtention of Spirulina biomasses particularly rich in γ-linolenic acid and/or in sulfolipids, which constitute an later object of invention.

For the Spirulina biomass rich in γ-linolenic acid, the proportion of γ-linolenic acid in the biomass compared with the total fatty acids is of at least about 25%, notably of about 29 to 35% for the culture in pond, and of about 30 to 36% for the culture in a photobioreactor, whereas with a classic process this proportion is only of about 20 to 22%.

For the Spirulina biomass rich in sulfolipids, the proportion of these sulfolipids compared with total lipids is at of least about 35%, notably of about 38 to 42% for the culture in pond and of 39-43% for the culture in a photobioreactor. Also, the percentage of γ-linolenic acid is slightly increased.

A slight increase of the content of total lipids in the produced biomasses rich in γ-linolenic acid or in sulfolipids is also observed.

The culture process of Spirulina rich in γ-linolenic acid according to the invention can be advantageously used in order to produce a biomass rich in this fatty acid under the form of a food supplement with high nutritive value for human or animals (for example laying hens, shrimps, deers, goats). Moreover, the extraction of an oil rich in γ-linolenic acid from Spirulina biomass can be carried out by well known methods [14] in order to provide a category of food oil containing 75 to 85% of γ-linolenic acid. The purification of γ-linolenic acid isolated from this oil can be efficiently performed because it contains only small contents of saturated and monounsaturated fatty acids. The finished product under the form of natural γ-linolenic acid can be used in pharmaceutical and cosmetic industry.

The invention is illustrated by the following examples which do not present any restrictive character. The figures referred to in the examples are as follows:

FIG. 1: diagram of the preparation of Spirulina precultures (preparation of inoculum) for mass production;

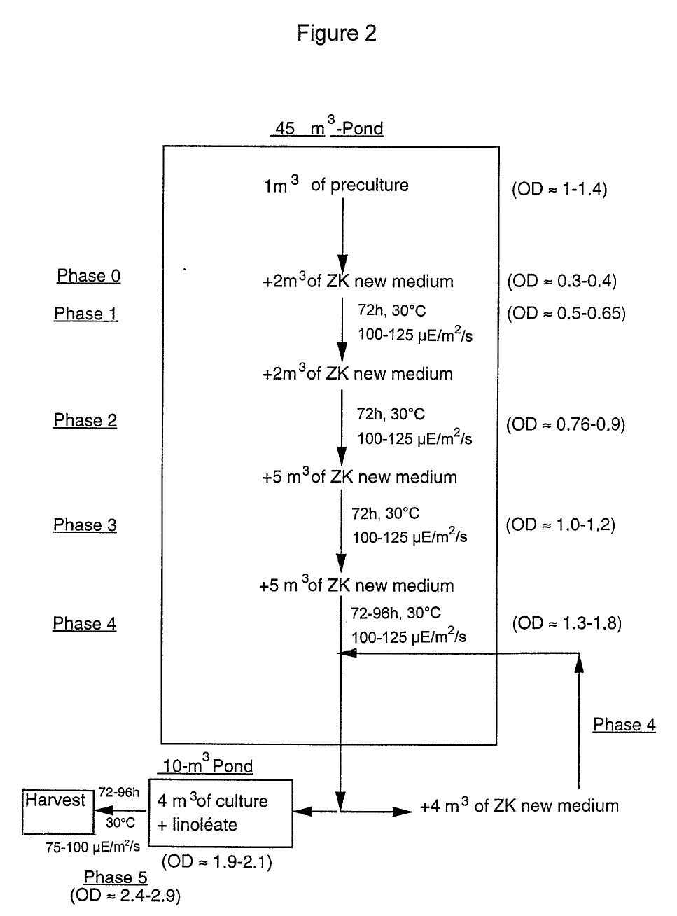

FIG. 2: diagram of the production process of Spirulina biomass rich in γ-linolenic acid in pond;

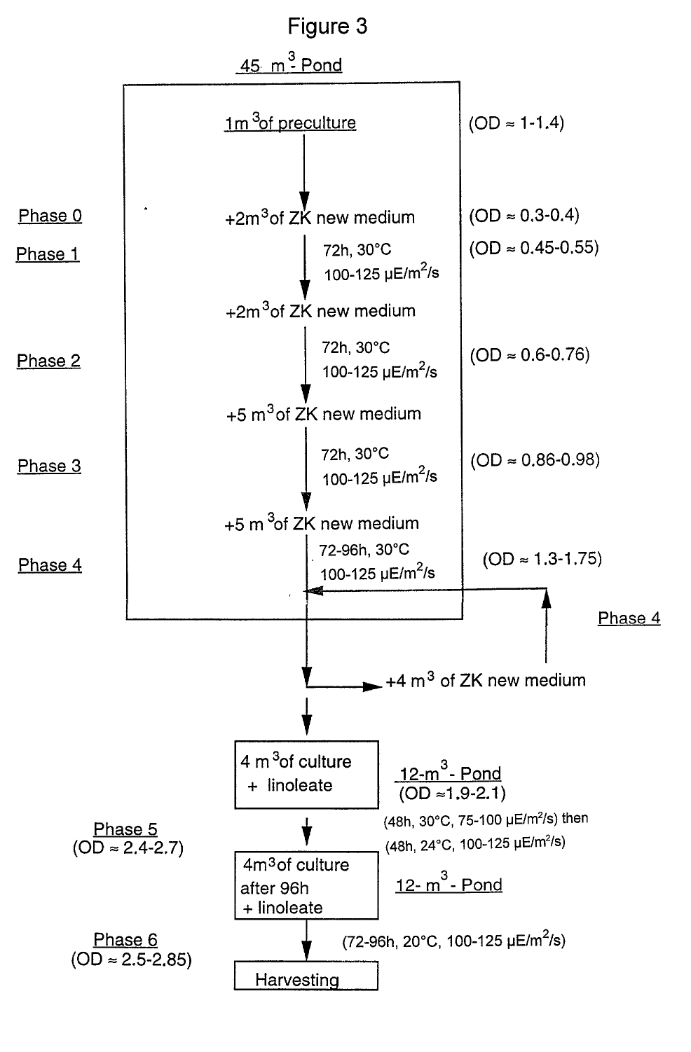

FIG. 3: diagram of the production process of Spirulina biomass rich in sulfolipids in pond;

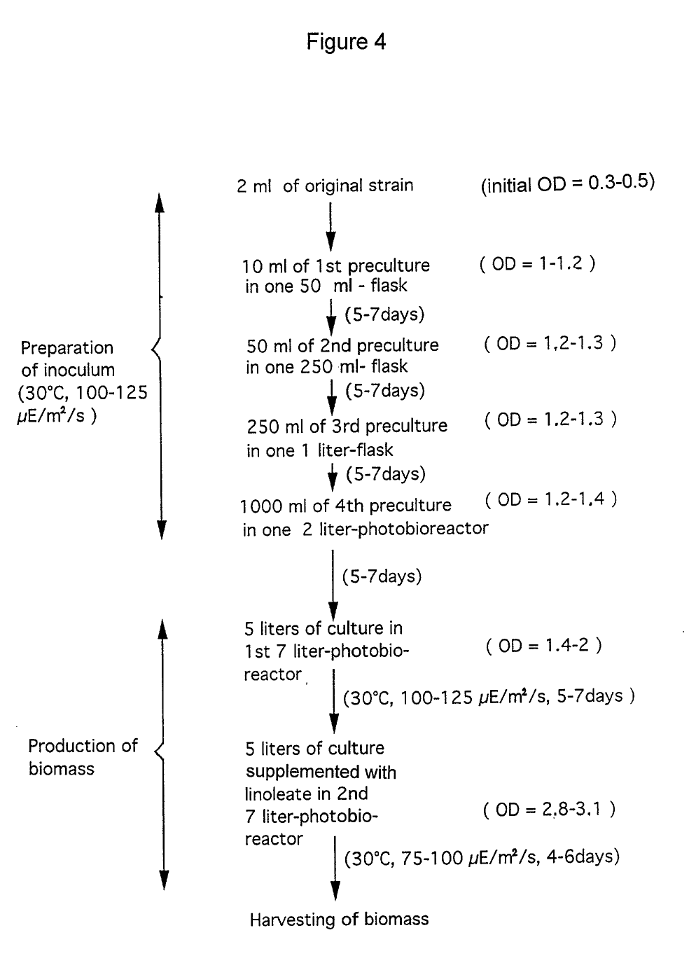

FIG. 4: diagram of the production of Spirulina biomass rich in γ-linolenic acid in a photobioreactor;

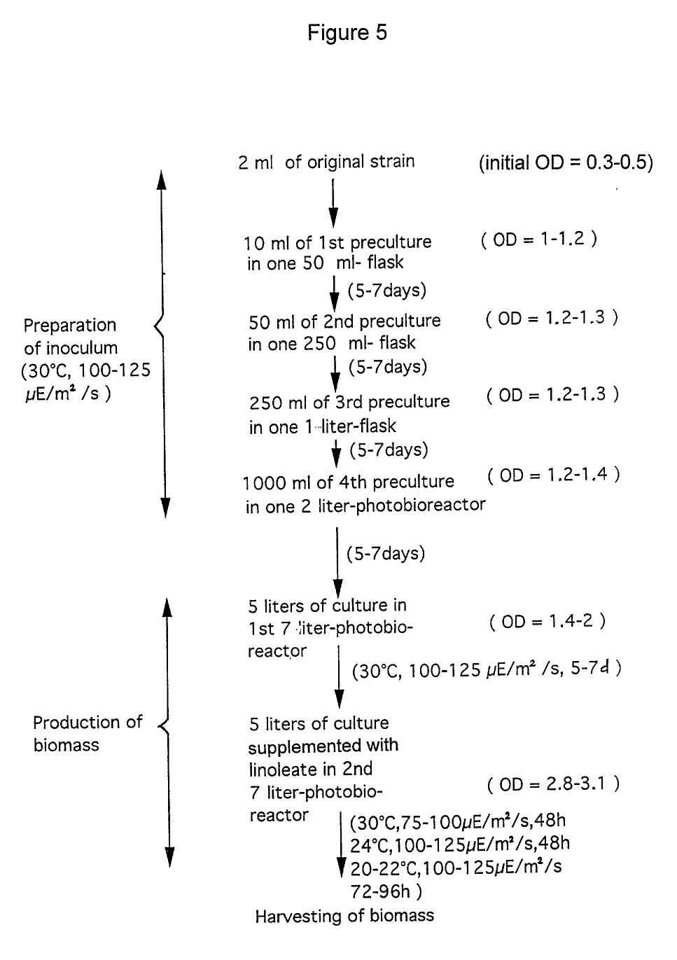

FIG. 5: diagram of the production of Spirulina biomass rich in sulfolipids in a photobioreactor.

### EXAMPLE 1

### Production of Spirulina Biomass Rich in γ-Linolenic Acid in Pond

1. Preparation of Original Strains and Precultures for Mass Production:

1.1. Original Strains:

The 5 following strains of Spirulina were used:

Spirulina platensis PCC 8005 from Institut Pasteur Paris;

Spirulina maxima and Spirulina texcoco (Texcoco, Mexico);

Spirulina crater (Laboratoire La Roquette, France).

They were purified and preserved in Zarrouk's liquid or solid medium (Table 4), at 20-22° C. and under low alternate illumination (50 to 75 μE/m²/s) with 16 h of light/8 h in obscurity.

1.2. Precultures:

The precultures in 1 liter- and 5 liter-flasks are prepared and pricked every 15 days. The pH of Zarrouk's medium is adjusted to 8.5 before sterilization at 120° C. during 20 min. The precultures can be started by the initial cellular concentration at the optical density of 0.3 to 0.5 at 560 nm as measured with a spectrophotometer Varian DMS-90.

These precultures are aerated by bubbling air which also allows the supply in CO₂ with a flow rate of the gazeous mixture at 1% of CO₂ of 60-80 l/l of culture/h. Moreover, the precultures in small flasks (500 ml, 1 l, 2 l) can also be carried out in a CO₂ Gallemkamp incubator in which an atmosphere at 1% of CO₂ is maintained, with an orbital agitation of about 100 rounds/min. The illumination is of 100 μE/m²/s for all precultures.

The inoculum prepared in a 5 liter-flask will be used for the multiplication of strains with 150 liter-plastic sacks or 100 liter- and 500 liter-ponds in order to obtain 1000 liters of preculture, which are transferred to a 45 m³-pond in order to start the mass production (see FIG. 1).

2. Preparation of Culture Medium for Mass Production in Pond:

All precultures for seeding were prepared with Zarrouk's medium while the mass culture from 1 to 20 m³ were realised with ZK(1) medium (Table 5). The Zarrouk's medium (sterilized at 120° C., pH≈8.5-9 during 20 minutes and cooled to 30° C.) was used in order to prepare precultures of Spirulina (1st to 4th preculture). For other precultures (5th and 7th preculture) and mass production, ZK (1) new medium can be used to replace Zarrouk's medium.

2.1. Sterilization of Usual Water for Mass Culture:

The usual water can be sterilized by the ultrafiltration method with a Millipore Filter system or by sterilization with sodium hypochlorite: 20 ml of sodium hypochlorite are added to 140 liters of usual water which are maintained at 25-30° C. during 12-24 h. Then 15.4 ml of a solution of sodium thiosulfate (350 g/l) are added. The excessive quantity of chlorine can be eliminated by a bubbling of sterile air during 30-60 minutes.

Sea water can also be used by adding 40 ml of sodium hypochlorite to 140 liters of sea water.

2.2. Preparation of Ammonium Linoleate:

The ammonium linoleate can be synthesized by the 2 following methods:

(a) Method 1:

4 ml of ammoniacal ethyl ether, prepared by bubbling anhydrous ammonia during 1 minute in a tube containing 10 ml of ethyl ether placed in ice, are added in a tube containing 0.5 g of dry linoleic acid.

The mixture is evaporated to dryness under nitrogen. The residue is re-solved in diluted buffer (for example tris-HCl buffer 10 mM) or in water buffered at pH≈8-9 by ammonia in order to avoid their dissociation. After rinsing the tube walls, buffered water is added to obtain a final volume of about 50 ml.

(b) Method 2:

5.4 ml of a 18% ammonia solution and 4.6 ml of bidistilled water are added to 0.5 g of dry linoleic acid. The mixture is heated at 50-60° C. during 30-45 minutes. The pH is adjusted to 8-9 with bidistilled water after elimination of excessive ammonia with gazeous nitrogen. The final volume of ammonium linoleate solution is about 50 ml. The concentration of this ammonium linoleate solution is 10 mg/ml.

The solution of ammonium linoleate is sterilised by filtration on a Millipore® filter 0.1-0.2 μm under sterile conditions. Moreover, silicone 426R (at a concentration of 1 mg/l, pH 6) is used as an antifoam in order to eliminate foams at the surface of the culture medium.

3. Mass production of Spirulina rich in γ-linolenic acid in pond (FIG. 2):

(a) After the preparation of 1 m³ of inoculum (7th preculture) with the initial cellular concentration (O.D. at 560 nm 1-1.2), a 45 m³-pond equipped with a paddlewheel in order to assure the circulation of culture medium with a speed about from 10 to 20 cm/s; a thermoregulator to maintain culture temperature at 30° C. and regulate the culture temperature at 20 to 35° C. according to the need of the culture process; a system of illumination in order to ensure an illumination of 50 to 125 μE/m²/s and a centrifugal pump in order to supply water and harvest Spirulina biomass was used.

The depth of the culture medium can be adjusted to 20 to 40 cm. The ammonium linoleate and ZK (1) new medium were prepared separately at pH 8.5-9. The clean pond was installed under a plastic greenhouse in order to avoid the contamination and use effectively sun light for the production of biomass.

(b) The Process of Production Comprises the Following Phases:

Phase 0: 1 m³ of inoculum (7th preculture) having an O.D. 1-1.2 is prepared. The 45 m³-pond is washed and the cleanliness of this pond is checked before inoculation. Then, 2 m³ of ZK (1) new medium are prepared and the pH is adjusted at 8.5-9.

Phase 1: 1 m³ of inoculum is added to 2 m³ of ZK(1) new medium having an O.D. of ≈0.3-0.4. The temperature is set and maintained at 30° C. and light intensity at 100-125 μE/m²/s. The speed of circulation of the culture medium (V) is maintained at 10 cm/s. After 72 h, the O.D.≈0.55-0.65 is measured to determine the growth rate of Spirulina.

Phase 2: 3 m³ of culture (of phase 1) are added to 2 m³ of ZK (1) new medium. The culture is maintained at 30° C., 100-125 μE/m²/s, pH≈9, V≈10 cm/s during 72 h. The O.D.≈0.76-0.9 is measured in order to determine the growth of Spirulina.

Phase 3: 5 m³ of ZK (1) new medium are added to 5 m³ of culture (of phase 2) and all culture conditions of phase 2 are maintained. After 72 h, the measured O.D. is 1.1-1.25.

Phase 4: 5 m³ of ZK (1) new medium are added to 10 m³ of culture (of phase 3) and all culture conditions of phase 3 are maintained. After 72-96 h, the measured O.D. is 1.4-1.8.

Phase 5: 4 m³ of culture of phase 4 are extracted and transferred to the second 12 m³-pond in order to continue the culture of Spirulina in the presence of ammonium linoleate (35-75 μM linoleate/liter). The measured O.D. is 1.9-2.1. Simultaneously, 4 m³ of ZK (1) new medium are added to 11 m³ of culture of phase 4 in the principal pond in order to prepare another culture of phase 4 while checking the medium composition and re-complementing all nutritive elements which are necessary to the growth of Spirulina.

The culture supplemented with linoleate is maintained at 30° C., with an illumination of about 75-100 μE/m²/s and pH 9-10; after 72-96 h, the measured O.D. is ≈2.4-2.9.

(c) Culture Conditions:

The culture conditions such as pH, temperature, illumination, bubbling with air enriched with 1% of CO₂ mentioned above in the description have to be maintained.

Also, maintaining the ion concentrations adapted to the needs of Spirulina during the growth should be taken care of, as also indicated above.

(d) Harvest of the Spirulina Biomass:

The culture of Spirulina is maintained in decanting tanks at 24° C. during 24 h in order to eliminate the supernatant. The biomass precipitates at the bottom of tanks and is harvested by filtration or centrifugation at 5000 rounds/min during 15 minutes, then rinsed with a solution of NaCl at concentration of 10 g/l at 24° C. The biomass is then harvested by a new centrifugation at 5000 rounds/min during 15 minutes and finally rinsed 3 times with distilled or demineralized water before lyophilization or spraying.

4. Results:

The general chemical composition of Spirulina cultivated by the process of invention for obtaining the biomass rich in γ-linolenic acid, their lipid composition and their total fatty acid composition are reported respectively in tables 6, 7 and 8 below.

TABLE 6
General chemical composition of Spirulina strains (%) cultivated in
pond in order to obtain a biomass rich in γ-linolenic acid.

| | Sp. 8005 | Sp. Maxima | Sp. Texcoco | Sp. crater |
|---|---|---|---|---|
| Proteins | 55-60 | 54-59 | 53-58 | 56-58 |
| Carbohydrates | 15-18 | 13.5-15.0 | 10-12 | 11-13 |
| Lipids | 6.8-7.4 | 6.8-7.5 | 7.0-7.5 | 6.7-7.4 |
| Minerals | 7.0-12.0 | 7.0-8.5 | 8-11 | 7.0-8.5 |
| Humidity | 7.0-8.0 | 7.0-8.0 | 7.0-8.0 | 7.0-8.0 |
| Carotenoids | 0.20-0.29 | 0.30-0.35 | 0.15-0.20 | 0.15-0.19 |
| Chlorophyll a | 0.7-0.8 | 0.67-0.72 | 0.70-0.78 | 0.7-0.8 |

TABLE 7
Lipid composition of Spirulina strains (%) cultivated in pond in order
to obtain biomasses rich in γ-linolenic acid.

| | Sp. 8005 | Sp. Maxima | Sp. Texcoco | Sp. crater |
|---|---|---|---|---|
| MGDG | 46.1-47.5 | 44.8-46.8 | 45.0-46.3 | 42.8-44.5 |
| DGDG | 10.7-12.3 | 11.0-11.7 | 10.6-11.2 | 11.5-11.7 |
| PG | 19.0-20.4 | 18.9-19.5 | 18.8-19.6 | 19.5-20.1 |
| SQDG | 21.5-22.5 | 21.7-22.4 | 22.5-22.8 | 21.2-23.7 |

TABLE 8
Total fatty acid composition of Spirulina strains (%) cultivated in
pond in order to obtain a biomass rich in γ-linolenic acid.

| | Sp. 8005 | Sp. Maxima | Sp. Texcoco | Sp. crater |
|---|---|---|---|---|
| Palmitic acid | 30 | 35 | 34.5 | 36.6 |
| (C16:0) | | | | |
| Palmitoleic acid | 1.3 | 1.2 | 1.3 | 1.2 |
| (C16:1) | | | | |
| Stearic acid | 1.4 | 1.4 | 1.4 | 1.4 |
| (C18:0) | | | | |
| Oleic acid | 3.7 | 4.8 | 4.7 | 4.5 |
| (C18:1) | | | | |
| Linoleic acid | 28.8 | 27.9 | 28.4 | 27.2 |
| (C18:2) | | | | |
| γ-linolenic acid | 34.8 | 29.7 | 29.7 | 29.1 |
| (γ-C18:3) | | | | |
| ΣC16/ΣC18 | 31.3/68.7 | 36.2/63.8 | 35.8/64.2 | 37.8/62.2 |

The results show that:

The produced biomasses contain from 6.7 to 7.5% of dry weight under the form of lipids, with an increase as compared to original strains. Moreover, the carotenoid content also increases and reaches from 0.15 to 0.35% [Table 6]. The obtained biomasses contain an important quantity of MGDG reaching from 42.8 to 47.5% of the total lipids [Table 7] while the proportion of SQDG is only about from 21.2 to 23.7% of the total lipids. Moreover, the content of γ-linolenic acid in this biomass increases significantly and reaches from 29 to 34.8% of total fatty acids;

The accumulation of γ-linolenic acid goes together with an increase of linoleic acid in these biomasses [Table 8], showing that the exogenous linoleic acid under the form of ammonium linoleate was absorbed and desaturated by the Δ⁶ desaturase in order to form γ-linolenic acid in Spirulina cells;

The percentages of total C16 fatty acids in the biomasses decrease according to the increase of C18 total fatty acids (31.3/68.7 in Spirulina 8005).

### EXAMPLE 2

### Production of Spirulina Biomass Rich in Sulfolipids in Pond

1. The preparation of original strains, precultures and the preparation of the culture medium are as described in example 1.

2. The production process of which the diagram is shown on FIG. 3, comprises the 6 following phases:

Phase 0 to phase 4 (in the first 45 m³-pond): Spirulina was cultivated as described in example 1. At the end of phase 4, 4 m³ of culture of phase 4 are taken and transferred to the second pond, then 4 m³ of ZK (1) new medium are added to 11 m³ of culture of phase 4 while simultaneously controlling the medium composition and re-complementing the medium with nutritive elements necessary to the growth of Spirulina.

Phase 5 (in the second 12 m³-pond): 4 m³ of Spirulina supplemented with ammonium linoleate at the linoleate concentration of 35 to 75 μM/liter of culture medium are cultivated at 30° C./48 h, then at 24° C. during 48 h, under an Ilumination of about 75-125 μE/m²/s. After 96 h of culture, this culture volume is transferred to the 3rd pond in order to stimulate the synthesis of sulfolipids in Spirulina cells. Simultaneously, a new volume of culture (4 m³) is taken from the first pond in order to prepare another culture in the presence of ammonium linoleate.

The cellular concentration reaches an O.D. at 560 nm of ≈2.4-2.7 at the end of phase 5.

Phase 6 (in the 3rd 12 m³-pond): the culture of Spirulina supplemented in linoleate at 20-22° C. under illumination from 100 to 125 μLE/m²/s is continued during 72-96 h before harvesting the biomass. The cellular concentration reaches an O.D. at 560 nm of ≈2.5-2.8 at the end of phase 6.

The harvest is carried out under the conditions described in example 1.

3. Results:

The general chemical composition of Spirulina cultivated by the process of the invention for obtaining of a biomass rich in sulfolipids, their lipid composition and their total fatty acid composition are respectively reported in tables 9, 10 and 11 below.

TABLE 9
General chemical composition (%) of Spirulina strains cultivated
in pond in order to obtain biomasses rich in sulfolipids.

| | Sp. 8005 | Sp. Maxima | Sp. Texcoco | Sp. crater |
|---|---|---|---|---|
| Proteins | 54-59 | 53-60 | 53-59 | 56.0-59.5 |
| Carbohydrates | 13-16 | 13.0-14.8 | 10.0-11.5 | 12.0-13.4 |
| Lipids | 6.7-7.2 | 6.6-7.2 | 6.8-7.1 | 6.9-7.0 |
| Minerals | 7.0-10.8 | 7.3-8.2 | 8.0-11.0 | 7.4-8.5 |
| Humidity | 7.0-8.0 | 7.2-8.0 | 7.0-8.0 | 7.3-8.2 |
| Carotenoids | 0.16-.024 | 0.28-0.32 | 0.12-0.17 | 0.13-0.18 |
| Chlorophyll a | 0.7-0.8 | 0.7-0.8 | 0.73-0.78 | 0.72-0.77 |

TABLE 10
Lipid composition (%) of Spirulina strains cultivated in
pond in order to obtain biomasses rich in sulfolipids.

| | Sp. 8005 | Sp. Maxima | Sp. Texcoco | Sp. crater |
|---|---|---|---|---|
| MGDG | 34.0-34.8 | 34.5-35.6 | 37.0-38.4 | 36.9-38.1 |
| DGDG | 9.5-10.2 | 9.0-9.5 | 8.8-9.6 | 8.6-9.2 |
| PG | 12.9-13.4 | 14.0-14.6 | 12.0-12.8 | 13.6-14.0 |
| SQDG | 40.8-41.6 | 39.5-40.3 | 38.5-39.2 | 38.0-38.7 |

TABLE 11
Total fatty acid composition (%) of Spirulina strains cultivated in pond in
order to obtain biomasses rich in sulfolipids.

| | Sp. 8005 | Sp. Maxima | Sp. Texcoco | Sp. crater |
|---|---|---|---|---|
| Palmitic acid | 40.2 | 41.0 | 41.5 | 41.1 |
| (C16:0) | | | | |
| Palmitoleic acid | 2.7 | 2.8 | 2.5 | 3.3 |
| (C16:1) | | | | |
| Stearic acid | 3.0 | 3.3 | 3.2 | 3.8 |
| (C18:0) | | | | |
| Oleic acid | 3.4 | 3.8 | 4.9 | 4.7 |
| (C18:1) | | | | |
| Linoleic acid | 24.9 | 24.4 | 22.2 | 21.4 |
| (C18:2) | | | | |
| γ-linolenic acid | 25.8 | 25.1 | 25.7 | 25.7 |
| (γ-C18:3) | | | | |
| ΣC16/ΣC18 | 42.9/57.1 | 43.8/56.2 | 44/56 | 44.4/55.6 |

The results show that:

The Spirulina strains contain about from 6.6 to 7.2% of dry weight under the form of lipids, with an increase as compared to original strains [Table 9];

The percentage of MGDG decreases according to the increase of SQDG (or sulfolipids). The proportion of sulfolipids significantly increases in all used strains and varies between 38 and 41.6% of total lipids while the percentage of MGDG in all used strains decreases and varies between 34 and 38.4% of total lipids [Table 10]. These results prove that decreasing the temperature (30° C.→24° C.→20° C.) stimulates the synthesis of sulfolipids;

The proportion of γ-linolenic acid slightly increases from 20.4 to 25.8% in Spirulina 8005;

The ratio of C16 total fatty acids/C18 total fatty acids does not significantly vary and is only about 42.9/57.1 in Spirulina 8005 [Table 11].

These results show that the culture conditions of the process of the invention significantly influence the proportion of sulfolipids in the biomass.

### EXAMPLE 3

### Culture Process of Spirulina Rich in γ-Linolenic Acid in a Sterile Photobioreactor (FIG. 4)

1. Multiplication of Spirulina Strains:

The same strains as in example 1 are used.

All precultures of Spirulina were prepared by cultures in flasks (50 ml, 250 ml and 1 liter) and in a 2 liter-photobioreactor.

The preparation of precultures is carried out once at the beginning of mass production. The strains multiplication is carried out by the 4 following stages:

(a) The first preculture (100 ml) is prepared with a 50 ml-flask placed in a Gallemkamp incubator, with an orbital agitation of 100 to 200 rotations/min, in which the atmosphere is maintained at 1% of CO₂, under an illumination of 100 μE/m²/s. The cycle of illumination is about 12 h of white light/12 h in obscurity during 5-7 days at 30° C. The initial cellular concentration is about 0.2-0.35 g dry/liter of Zarrouk's medium (O.D.₅₆₀≈0.3-0.5) and the final cellular concentration of the 1st preculture reaches an O.D.₅₆₀ of ≈1-1.2.

(b) 10 ml of the 1st preculture are used as inoculum in order to make the second preculture in a 250 ml-flask containing 40 ml of Zarrouk's sterile medium, placed in a Gallemkamp incubator under the culture conditions used for the first preculture. After 5-7 days of culture, 50 ml of the 2nd preculture with an O.D.₅₆₀ of ≈1.2-1.3 are obtained.

(c) 50 ml of the 2nd preculture are added to 200 ml of Zarrouk's sterile medium in order to begin the 3rd preculture. This preculture is carried out in a 1 liter-flask placed in a Gallemkamp incubator under the culture conditions used for the first preculture. After 5-7 days of culture, 50 ml of the 3rd preculture with an O.D.₅₆₀ of ≈1.2-1.3 are obtained.

(d) In order to carry out the 4th preculture, 1 liter of Zarrouk's sterile medium is prepared in a sterile 2 liter-photobioreactor. Then 250 ml of the 3rd preculture are added into this photobioreactor placed under the culture conditions of the first preculture. The bubbling of sterile air enriched with 1% of CO₂ is adjusted to a flow rate of 50 to 70 l/l of culture/h. After 5-7 days of culture, 1 liter of the 4th preculture with an O.D.₅₆₀ of ≈1.2-1.4 can be obtained.

2. Production of a Biomass of Spirulina Rich in γ-Linolenic Acid in Photobioreactor (FIG. 4).

(a) 4 liters of Zarrouk's sterile medium are prepared in a 7 liter-photobioreactor while adding 1 liter of 4th preculture as inoculum prepared at the stage 1.d). The culture is carried out under the same culture conditions used for the previous precultures (O.D.₅₆₀≈1.4-2.0 after 5-7 days of culture), with an agitation speed of about 100-150 rounds/min. The bubbling of sterile air enriched with 1% of CO₂ is adjusted to a flow rate of 50 to 70 l/l of culture/h.

(b) 4 liters of culture are taken in the 1st photobioreactor while re-complementing the medium with 4 liters of Zarrouk's sterile new medium in order to continue preparing the next culture. Then 4 liters of this culture as sterily transferred to the second 7 liter-photobioreactor while supplementing the medium with ammonium linoleate (at a concentration of 35 to 75 μM linoleate/l) in the middle of the exponential phase of growth during 4-6 days. The culture is carried out under the following conditions: at 30° C., under an illumination of 75 to 100 μE/m²/s, with a cycle of illumination by 24 h of 8-12 h in white light/16-12 h in obscurity after the supplementation with linoleate. Simultaneously, the bubbling of sterile air enriched with 1% of CO₂ is adjusted to 20-30 l/l of culture/h after the supplementation with linoleate and maintained during 24 h in order to avoid the formation of foams at the surface of culture medium. Silicon 426R is used as antifoam. The agitation speed is maintained at about 100-150 rounds/min. Then, the bubbling of sterile air enriched with 1% of CO₂ has to be increased and maintained between 50 and 70 l/l of culture/h.

(c) After 4-6 days of culture, the biomass is harvested under the conditions described in example 1.

In this example, the culture was carried out with a higher cellular concentration. This allows decreasing the duration of production while increasing the culture yield (from 2.4 to 2.6 g dry per liter).

3. Results

Spirulina strains cultivated in a photobioreactor according to the process of the invention contain a more important amount of γ-linolenic acid more important than those cultivated in a pond. The proportion of γ-linolenic acid of the obtained biomass is about 30-36% of total fatty acids (Tables 12 and 13). The culture yield in a photobioreactor is higher than that of culture in pond and reaches 1.78-2.2 g dry per liter.

TABLE 12
Lipid composition (%) of Spirulina strains cultivated in a photobioreactor
in order to obtain biomasses rich in γ-linolenic acid.

| | Sp. 8005 | Sp. Maxima | Sp. Texcoco | Sp. crater |
|---|---|---|---|---|
| MGDG | 45.8-47.8 | 45.1-47.0 | 45.3-46.6 | 43.1-44.8 |
| DGDG | 11.0-11.5 | 11.3-11.8 | 11.0-11.5 | 11.6-11.8 |
| PG | 18.0-18.5 | 19.1-20.0 | 19.0-19.3 | 19.6-20.0 |
| SQDG | 21.4-22.2 | 20.7-21.2 | 22.4-22.6 | 21.0-23.4 |

TABLE 13
Total fatty acid compositions (%) of Spirulina strains cultivated in a
photobioreactor in order to obtain biomasses rich in γ-linolenic acid.

| | Sp. 8005 | Sp. Maxima | Sp. Texcoco | Sp. crater |
|---|---|---|---|---|
| Palmitic acid | 29.7 | 34.3 | 34.0 | 36.0 |
| (C16:0) | | | | |
| Palmitoleic acid | 1.2 | 1.2 | 1.3 | 1.2 |
| (C16:1) | | | | |
| Stearic acid | 1.3 | 1.4 | 1.4 | 1.3 |
| (C18:0) | | | | |
| Oleic acid | 3.5 | 4.6 | 4.5 | 4.2 |
| (C18:1) | | | | |
| Linoleic acid | 28.3 | 28.0 | 28.6 | 27.3 |
| (C18:2) | | | | |
| γ-linolenic acid | 36.0 | 30.5 | 30.2 | 30.0 |
| (γ-C18:3) | | | | |
| ΣC16/ΣC18 | 30.9/69.1 | 35.5/64.5 | 35.3/64.7 | 37.2/62.8; |

### EXAMPLE 4

### Culture Process of Spirulina Rich in Sulfolipids in a Sterile Photobioreactor (FIG. 5)

1. Production of a Biomass of Spirulina Rich in Sulfolipids in a Photobioreactor.

The same strains as in example 1 are used.

(a) The multiplication of strains is carried out in 4 stages as indicated in example 3. 1. The preparation of precultures is carried out only once at the beginning of mass production.

(b) The preparation of 5 liters of culture in the first 7 liter-photobioreactor is carried out according to the stages cited in example 3, 2), a).

(c) 4 liters of culture of the first photobioreactor are taken while re-complementing 4 liters of Zarrouk's sterile new medium in this photobioreactor in order to continue preparing another culture. Then, 4 liters of this culture are sterily transferred as inoculum to the second 7 liter-photobioreactor while supplementing the medium with ammonium linoleate (at the concentration of 35-75 μM linoleate/liter) during 5-7 days, under the following culture conditions:

c.1) the first stage comprises 2 successive phases:

Phase 1: the culture is maintained at 30° C. under an illumination of 75 to 100 μE/m²/s during 48 h. Simultaneously, the illumination cycle should be adjusted to about 8-12 h of white light/16-12 h in obscurity by 24 h. Moreover, the bubbling of sterile air enriched with 1% of CO₂ must be lowered and maintained at the flow rate of 25 to 35 l/l of culture/h during 24 to 48 h. The agitation speed is maintained at about 100-150 rounds/minute.

Phase 2: the culture is placed at 24° C. under a higher illumination of 100 to 125 μE/m²/s during 48 h. The cycle of illumination is of about 8-12 h of white light/16-12 h in obscurity by 24 h. The bubbling of sterile air enriched with 1% of CO₂ is increased and maintained at the higher flow rate of 40 to 50 l/l of culture/h during 48-72 h. The agitation speed is maintained at about 100-150 rounds/min.

c.2) In the second stage, the culture temperature is lowered to 20-22° C., under an illumination of 100-125 μE/m²/s. The cycle of illumination is of about 12 h in white light/12 h in obscurity during 72-96 h before harvesting the biomass. The pH is about 9-10.5 in order to optimize the synthesis of sulfolipids in Spirulina cells. The culture is aerated with a mixture of sterile air enriched with 1% of CO₂ at the flow rate of 50-60 l/l of culture/h. The agitation speed is maintained at about 100-150 rounds/min. The duration of the second stage can vary according to the used strains.

d) The biomass rich in sulfolipids is harvested by the harvesting process indicated in example 1. The diagram of the production of Spirulina rich in sulfolipids in a photobioreactor is shown on FIG. 5. The increase of the initial cellular concentration makes it possible to reduce the culture duration while increasing the production yield (from 2.3 to 2.6 g dry/liter).

2. Results

Spirulina produced in photobioreactor by the process of invention contain an important amount of sulfolipids reaching 39-43% of total lipids (Tables 14 and 15). The culture yield reaches from 1.65 to 2.1 g dry/liter.

TABLE 14
Lipid composition (%) of Spirulina strains cultivated in a photobioreactor
in order to obtain biomasses rich in sulfolipids

| | Sp. 8005 | Sp. Maxima | Sp. Texcoco | Sp. crater |
|---|---|---|---|---|
| MGDG | 33.8-34.9 | 34.0-35.2 | 36.8-37.3 | 36.5-37.4 |
| DGDG | 9.8-10.8 | 9.2-9.6 | 8.9-9.2 | 8.6-9.1 |
| PG | 11.0-11.3 | 13.0-14.0 | 12.4-12.8 | 12.6-13.0 |
| SQDG | 40.9-43.0 | 39.8-41.2 | 39.2-40.7 | 39.0-40.5 |

TABLE 15
Total fatty acid composition (%) of Spirulina strains cultivated in a
photobioreactor in order to obtain biomasses rich in sulfolipids.

| | Sp. 8005 | Sp. Maxima | Sp. Texcoco | Sp. crater |
|---|---|---|---|---|
| Palmitic acid | 40.2 | 41.3 | 41.8 | 41.5 |
| (C16:0) | | | | |
| Palmitoleic acid | 2.7 | 2.6 | 2.4 | 3.1 |
| (C16:1) | | | | |
| Stearic acid | 3.2 | 3.4 | 3.2 | 3.6 |
| (C18:0) | | | | |
| Oleic acid | 3.4 | 3.7 | 4.8 | 4.5 |
| (C18:1) | | | | |
| Linoleic acid | 24.6 | 23.7 | 22.0 | 21.7 |
| (C18:2) | | | | |
| γ-linolenic acid | 25.9 | 25.3 | 25.8 | 25.6 |
| (γ-C18:3) | | | | |
| ΣC16/ΣC18 | 42.9/57.1 | 43.9/56.1 | 44.2/55.8 | 44.6/55.4 |

### EXAMPLE 5

### Effect of the Additional Supplementation in Glucose

Spirulina platensis PCC 8005 was cultivated in a photobioreactor in Zarrouk's medium supplemented with ammonium linoleate, or with ammonium linoleate and glucose.

The culture in photobioreactor is carried out under the following conditions:

The precultures in the presence of glucose are incubated in obscurity under a speed of agitation of 100 rounds/min during 10-14 days at 30° C. 250 ml-flasks containing 100 ml of Zarrouk's sterile medium are used to prepare these precultures while the mixotrophic culture is carried out in a photobioreactor. The initial cellular concentration (O.D.at 560 nm) is about 0.5-0.8.

Then, the culture in photobioreactor is placed at 30° C. under a continuous illumination of 50 to 100 μE/m²/s during 5-7 days. Finally, the culture is maintained at 30° C. under an illumination of 50-100 μE/m²/s with 8-12 h of white light/16-12 h in obscurity by 24 h during 5-7 days.

The harvest of biomass is carried out at the end of the obscurity period.

In the case of glucose supplementation, the exogenous glucose was used as a source of carbon in order to synthesize organic substances in Spirulina cells. The optimum concentration of glucose for the mixotrophic culture is about 2.5 to 3 g/l. The culture yield increases in terms of a glucose increase in the medium (from 0.5 to 2.5 g/l), then the yield of culture decreases slightly at higher concentration of exogenous glucose (from 5 g/l to 10 g/l). Finally, the culture yield reaches 2.5-2.7 g dry/liter.

The results are reported in tables 16, 17 and 18 below.

The lipid content is of about 7.7-9.2% of dry weight in the case of simultaneous supplementation in glucose and linoleate (Table 16). The cells supplemented either with glucose or with glucose and linoleate significantly synthesized MGlcDG (monoglucosyldiacylglycerol), precursor of MGDG and the proportion of MGlcDG reaches 10.2-12.2% of total lipids (Table 17).

Simultaneously, the synthesis of γ-linolenic acid was stimulated in these cells and the content of this fatty acid reaches 35.8% of total fatty acids (Table 18).

TABLE 16
General chemical composition (%) of Spirulina platensis PCC 8005
cultivated in Zarrouk's medium supplemented with linoleate (35.7 μM/1)
or with (2.5 g/l) and linoleate (35.7 μM/l) at 30° C.

| | + Linoleate | + Glucose & linoleate |
|---|---|---|
| Proteins | 55-60 | 57-62 |
| Carbohydrates | 15-18 | 15-19 |
| Lipids | 6.8-7.4 | 7.7-9.2 |
| Minerals | 7.0-12.0 | 3.7-5.5 |
| Humidity | 7.0-8.0 | 6-7 |
| Carotenoids | 0.20-0.29 | 0.2-0.3 |
| Chlorophyll a | 0.7-0.8 | 0.68-0.76 |

TABLE 17
Lipid composition in Spirulina platensis PCC 8005 cultivated in Zarrouk's
medium supplemented with linoleate (35.7 μM/l) or with glucose (2.5 g/l)
and linoleate (35.7 μM/l) at 30° C.

| | Polar lipids (%) | | | | |
|---|---|---|---|---|---|
| Culture conditions | MGlcDG | MGDG | DGDG | PG | SQDG |
| − Without supplementation | — | 31.5-33 | 15.7-17.3 | 24.8-26.2 | 24.8-26.6 |
| + Linoleate | — | 45.8-47.8 | 11.0-11.5 | 18.0-18.5 | 21.4-22.2 |
| + Glucose & linoleate | 11.6-12.2 | 43.0-44.0 | 11.7-12.3 | 13.0-13.6 | 18.9-19.7 |

TABLE 18
Total fatty acid composition in Spirulina platensis PCC 8005 cultivated in
Zarrouk's medium supplemented with linoleate (35.7 μM/l) or with
(2.5 g/l) and linoleate (35.7 μM/l) at 30° C.

| | Total fatty acids (%) | | | | | | |
|---|---|---|---|---|---|---|---|
| Culture conditions | C16:0 | C16:1 | C18:0 | C18:1 | C18:2 | γC18:3 | ΣC16/ΣC18 |
| − Without | 51.2 | 3.6 | 0.7 | 5.9 | 18.2 | 20.4 | 54.8/45/2 |
| supplementation | | | | | | | |
| + Linoleate | 29.7 | 1.2 | 1.3 | 3.5 | 28.3 | 36.0 | 30/9/69.1 |
| + Glucose & linoleate | 30.2 | 1.0 | 1.2 | 4.8 | 27.0 | 35.8 | 31.2/68.8 |

### REFERENCES

1. ANASUYA DEVI M. and VENKATARAMAN L. V., 1983b. Supplementary values of the proteins of the blue green alga Spirulina platensis to rice and wheat proteins. Nutr. Rep. Intern. 28, 1029-1935.

2. BUCAILLE P., 1990. Intérêt et efficacité de l'algue Spirulina dans l'alimentation des enfants présentant une malnutrition protéino-énergétique en milieu tropical. Thése de doctorat d'etat en Médecine générale, Université de Toulouse.

3. QUIDSIA AKHTER S., 1991. A study on recovery from malnutrition and anaemia using Spirulina. Paper presented at the 1 st seminar on “Use of Algae. A means to combat malnutrition” held at CESTI, Dhaka, on November 3rd, 1991.

4. NGUYEN Lan Dinh, 1995. “Evaluation du projet de lutte contre la malnutrition infantile” menée par le Centre de nutrition infantile de Hochiminh ville grâce à l'assistance du CESVI. Communication from Vietnam.

5. NGUYEN Lan Dinh, 1995. Steps of developing and researching Spirulina at Child nutrition center. Paper presented at the seminar on “Utilisation de l'algue Spiruline en nutrition et en thérapeutique”, held at Hochiminh city, Vietnam, on Apr. 12th, 1995.

6. PHAM QUOC Kiet and PASCAUD Marc, 1996. Effects of dietary γ-linolenic acid on the tissue phospholipid fatty acid composition and the synthesis of eicosanoids in rats. Ann. Nutr. Metab. 40, 99-108. 7. GUSTAFSON K. R., CARDELLINA J. H., FULLER H. R. W., WEISLOW O. S., REBECCA F. K., SNADER K. M., PATTERSON G. M. L. and BOYD M. R., 1989. AIDS-antiviral sulfolipids from cyanobacteria (Blue-green algae). J. Nat. Cancer Inst., MD, Vol. 81, 16, 1254-1258.

8. BUSSON F., 1970. “Spirulina platensis (Gom.) et Spirulina Geitleri J. de Toni. Cyanophycées alimentaires”. Service de santé, Parc du Pharo, 13 Marseille 7ème, 159p.

9. SOSA TEXCOCO, 1987. Communication from Sosa Texcoco, Mexique.

10. SCHREURS W. H. P., 1987. Results of analysis of samples of Spirulina from Burma. TNO-CIVO Institutes, Netherlands.

11. CYANOTECH, 1990. Communication from Cyanotech, Hawaï, USA.

12. VONSHAK A. and RICHMOND A., 1988. Mass production of the blue-green alga Spirulina: an overview. Biomass 15, 233-247.

13. CORNET J. F., 1989. Etude cinétique et énergétique d'un photobioréacteur. Thèse de doctorat de l'université de Paris-Sud, Orsay, France.

14. COHEN Z. and HEIMER Y. M., 1990. Production of polyunsaturated fatty acids (EPA, ARA and GLA) by the microalgae Porphyridium and Spirulina. In Microalgal Biotechnology, Cambridge University Press, 243-273.

1. Method for mixotrophic culture of Spirulina for producing a biomass rich in ω6 polyunsaturated fatty acids, particularly in γ-linolenic acid and/or in sulfolipids, characterized in that said production comprises at least one culture stage of Spirulina in the presence of ammonium linoleate at a concentration comprised between 30 and 90 μM/l, preferably from 35 to 75 μM/l.

2. Method according to claim 1, characterized in that the culture stage of Spirulina in the presence of ammonium linoleate is carried out at a temperature comprised between 20 and 35° C. and under an illumination comprised between 50 and 125 μE/m²/s, preferably between 75 and 100 μE/m²/s.

3. Method according to anyone of claim 1 or 2, characterized in that ammonium linoleate is added to the culture medium during the exponential phase of growth, Method in the middle of this phase.

4. Method according to any one of claims 1 to 3 for producing a biomass of Spirulina rich in γ-linolenic acid, characterized in that the temperature is comprised between about 24° C. and 35° C., advantageously about 30° C. and the illumination is comprised between 75 and 125 μE/m²/s, advantageously between 75 and 100 μE/m²/s.

5. Method according to any one of claims 1 to 4 for producing a biomass of Spirulina rich in sulfolipids, characterized in that the temperature is preferably comprised between about 20 and 25° C. and the illumination is comprised between 100 and 125 μE/m²/s.

6. Method according to any one of claims 1 to 5, characterized in that glucose is added to the culture medium, preferably during the exponential phase of growth, at a concentration of about 2 to 5 g/l, preferably 2.5 to 3 g/l.

7. Method according to claim 6, characterized in that the supplementation with glucose is carried out at a temperature about 30° C., under an illumination of about 50-100 μE/m²/s.

8. Method according to one of claims 2 to 7, characterized in that the illumination is carried out according to an alternate cycle of about 8-12 h of white light and 16-12 h in obscurity, preferably 12 h of white light and 12 h in obscurity.

9. Method according to any one of claims 1 to 8, characterized in that the biomass of Spirulina is produced in a pond or in a sterile photobioreactor.

10. Method according to any one of claims 1 to 9, for the mass production of a biomass of Spirulina rich in γ-linolenic acid and/or in sulfolipids, characterized in that the culture conditions comprise the following conditions:
bubbling 60 to 80 liters of sterile air enriched with 1% of CO₂/l of culture medium/h in the precultures;
maintaining the pH of the culture medium between 8.5 and 10.5, preferably of 9 to 10 during the growth;
maintaining the concentrations of hydrogenocarbonate, phosphate and nitrate, adapted to the needs of the Spirulina strain during the growth;
maintaining the culture temperature at 30° C. while ensuring an illumination of 100 to 125 μE/m²/s with about 8 to 12 h of white light and about 16 to 12 h in obscurity by 24 h during the growth, preferably about 12 h of white light and about 12 h in obscurity, then lowering the illumination at 75 to 100 μE/m²/s during the last phase of biomass production.

11. Method according to any one of claims 1 to 10, for producing a biomass of Spirulina rich in γ-linolenic acid in pond, characterized in that the production of said biomass comprises one stage of supplementation with linoleate under an illumination of 75 to 100 μE/m²/s, according to an alternate cycle of illumination by 24 h of about 8 to 12 h of white light and about 16 to 12 h in obscurity, preferably about 12 h of white light and about 12 h in obscurity, while maintaining the temperature at about 30° C. and lowering if necessary the bubbling of air enriched with 1% of CO₂ to a flow rate of 30-40 liters/l of culture/h during 24 h after said supplementation.

12. Method according to any one of claims 1 to 11, for producing a biomass of Spirulina rich in γ-linolenic acid in asterile photobioreactor, characterized in that the culture is carried out, after the incorporation of ammonium linoleate, under the following conditions:
a) temperature of 28 to 32° C., preferably about 30° C.;
b) illumination of 75 to 100 μE/m²/s according to an alternate cycle of illumination by 24 h of 8 to 12 h of white light/16 to 12 h in obscurity, preferably about 12 h of white light and about 12 h in obscurity, during 72 to 96 h;
c) bubbling of air enriched with 1% of CO₂ at a flow rate of 20 to 30 liters/l of culture/h during 24 h after the supplementation with ammonium linoleate.

13. Method according to any one of claims 1 to 10, for producing a biomass of Spirulina rich in sulfolipids in pond, characterized in that the production of said biomass comprises one stage of supplementation with ammonium linoleate, under an illumination of 75 to 100 μE/m²/s at about 30° C. during 48 h, then of 100 to 125 μE/m²/s at about 24° C. during 48-72 h according to an alternate cycle of illumination of about 8 to 12 h of white light and about 16 to 12 h in obscurity by 24 h, and, finally, of 100 to 125 μE/m²/s at about 20-22° C. during 72 to 96 h according to an alternate cycle of illumination of about 8-12 h of white light and about 16-12 h in obscurity, optionally with a bubbling of air enriched with 1% of CO₂ at a flow rate of 25 liters/l of culture/h during 24-48 h after the supplementation with ammonium linoleate, then of 40-60 l/l of culture/h during 48 to 96 h.

14. Method according to any one of claims 1 to 10 for producing a biomass of Spirulina rich in sulfolipids in a sterile photobioreactor, characterized in that the production of said biomass comprises one stage of supplementation with ammonium linoleate, under an illumination of 75 to 100 μE/m²/s at about 30° C. during 48 h, then of 100 to 125 μE/m²/s at about 24° C. during 48 to 72 h with an alternate cycle of illumination of about 8 to 12 h of white light and about 16 to 12 h in obscurity by 24 h, and, finally, of 100 to 125 HE/m²/s at about 20-22° C. during 72 to 96 h, according to an alternate cycle of illumination of about 8 to 12 h of white light and about 16 to 12 h in obscurity, optionally with a bubbling of air enriched with 1% of CO₂ at a flow rate about 25 l/l of culture/h during 24-48 h after the supplementation with ammonium linoleate, and then of 40-60 l/l of culture/h during 48 to 96 h.

15. Method according to any one of claims 1 to 14, characterized in that the used strain is chosen among Spirulina platensis, Spirulina maxima, Spirulina crater and Spirulina texcoco.

16. Biomass of Spirulina rich in γ-linolenic acid characterized in that the proportion of γ-linolenic acid in the biomass with respect to the total fatty acids content is at least about 25% and in that the total lipid content is increased in the biomass.

17. Biomass of Spirulina rich in sulfolipids characterized in that the proportion of said sulfolipids is at least about 35% with respect to total lipids.

18. Biomass of Spirulina rich in sulfolipids according to claim 17, characterized in that the total lipid content is increased in the biomass.

19. Culture medium for the mixotrophic culture of Spirulina, having one of following compositions:

| ZK (1) | | | ZK (2) | | |
|---|---|---|---|---|---|
| NaHCO₃ | 8.4 | g | NaHCO₃ | 8.4 | g |
| NaCl | 5.0 | g | NaCl | 4.5 | g |
| K₂HPO₄ | 0.2 | g | K₂HPO₄ | 0.2 | g |
| MgSO₄ · 7H₂O | 0.2 | g | NaNO₃ | 3.0 | g |
| KNO₃ | 2.5 | g | MgSO₄ · 7H₂O | 0.2 | g |
| FeSO₄ or | 0.01 | g | FeSO₄ | 0.01 | g |
| FeSO₄ · 7H₂O | 0.018 | g | H₂O | 1 | liter |
| H₂O | 1 | liter | | | |
| ZK (3) | | | ZK (4) | | |
| NaHCO₃ | 8.4 | g | NaHCO₃ | 8.4 | g |
| NaCl | 5.5 | g | NaCl | 5.5 | g |
| K₂HPO₄ | 0.3 | g | (NH₂)₂CO or (NH₄)₂SO₄ | 0.1 | g |
| MgSO₄ · 7H₂O | 0.2 | g | NH₄H₂PO₄ | 0.1 | g |
| (NH₂)₂CO or | 0.2 | g | K₂SO₄ | 0.2 | g |
| (NH₄)₂SO₄ | | | MgSO₄ · 7H₂O | 0.2 | g |
| FeSO₄ | 0.01 | g | FeSO₄ | 0.01 | g |
| H₂O | 1 | liter | H₂O | 1 | liter |
